(19) United States
(12) Patent Application Publication
(10) Pub. No.: US 2025/0368725 A1
(43) Pub. Date: Dec. 4, 2025

(54) ANTI-RCP ANTIBODY AND PREPARATION METHOD THEREOF

(71) Applicant: Ruijin Hospital Affiliated to Shanghai Jiao Tong University School of Medicine, Shanghai (CN)

(72) Inventors: Junna YE, Shanghai (CN); Zhuochao ZHOU, Shanghai (CN); Yijun YOU, Shanghai (CN); Yue SUN, Shanghai (CN); Fan WANG, Shanghai (CN); Chengde YANG, Shanghai (CN)

(21) Appl. No.: 18/878,534

(22) PCT Filed: Sep. 21, 2023
(86) PCT No.: PCT/CN2023/120276

§ 371 Date: Dec. 23, 2024

(30) Foreign Application Priority Data
Oct. 11, 2022 (CN) 202211244354.1

(51) Int. Cl. C07K 16/18

(52) U.S. Cl. CPC C07K 16/18; C07K 2317/14; C07K 2317/565

(57) ABSTRACT

An anti-RCP antibody and a preparation method thereof. The anti-RCP antibody includes a heavy chain variable domain and a light chain variable domain; the heavy chain variable domain including a heavy chain complementarity determining region HCDR1 consisting of the amino acid sequence of SEQ ID NO: 1, a heavy chain complementarity determining region HCDR2 consisting of the amino acid sequence of SEQ ID NO: 2, and a heavy chain complementarity determining region HCDR3 consisting of the amino acid sequence of SEQ ID NO: 3; the light chain variable domain including a light chain complementarity determining region LCDR1 consisting of the amino acid sequence of SEQ ID NO: 4, a light chain complementarity determining region LCDR2 consisting of the amino acid sequence of SEQ ID NO: 5, and a light chain complementarity determining region LCDR3 consisting of the amino acid sequence of SEQ ID NO: 6.

### CROSS REFERENCE TO RELATED APPLICATIONS

This application is a U.S. National Stage of International Application No. PCT/CN2023/120276 filed on Sep. 21, 2023, which claims priority to Chinese Patent Application No. 202211244354.1 filed on Oct. 11, 2022 under 35 U.S.C. § 119, the entire contents of all of which are hereby incorporated by reference.

### REFERENCE TO AN ELECTRONIC SEQUENCE LISTING

The contents of the electronic sequence listing (substitute sequence listing.xml; Size: 11,110 bytes; and Date of Creation: Dec. 18, 2024) is herein incorporated by reference in its entirety.

### TECHNICAL FIELD

The present invention is in the field of antibody technology, and specifically relates to an anti-RCP antibody and a preparation method thereof.

### BACKGROUND ART

Systemic lupus erythematosus (SLE) is a systemic autoimmune disease characterized by multisystem and organ damage accompanied by massive autoantibody production. In China, the number of patients with the disease is as high as 9.1 million, mostly found among young women. The disease progresses rapidly and can progress to multiple organ failure if not controlled. The 5-year survival rate is only 73%, making it one of the main diseases that endanger human life. As an autoimmune disease, the study of autoantibodies against SLE plays an important role in revealing its pathogenesis. Evidence from clinical studies reveals that many autoantibodies may emerge earlier than the onset of disease. It has been reported in the literature that multiple autoantibodies, such as anti-nuclear antibodies, anti-double-stranded DNA antibodies, and anti-Sm antibodies, may be detected in the serum of patients before the diagnosis of SLE disease and even 5 years before the first symptoms of SLE. Hundreds of autoantibodies have been detected in patients with SLE to date, most of which contribute to disease diagnosis. Some autoantibodies are pathogenic and associated with lupus nephritis, such as anti-dsDNA antibodies and anti-C1q antibodies.

There is an increase in cell apoptosis in patients with SLE, and apoptotic cells can affect the phagocytic function of macrophages through MAPK (including JNK, p38, and ERK1/2) signaling pathway. Normally, macrophages can promptly clear apoptotic cells to maintain equilibrium. Otherwise, the accumulated apoptotic cells may cause inflammation and autoimmune reactions. Early apoptotic cells may express “cleared” signals, such as phosphatidylserine (PS) and phosphatidyl ethanolamine (PE). When the clearance of apoptotic cells is impaired, excess apoptotic cells may cause secondary apoptosis and release danger signals to damage the immune system. These danger signals include nuclear antigens, DNA, RNA, etc., which activate the NF-Kβ pathway and inflammasomes to promote inflammation, increase the production of IL-6 and IL-1β, which in turn activates T and B lymphocytes and further increase the production of autoantibodies against these antigens.

In vivo cell apoptosis and phagocytosis is an organic process that maintains tissue integrity and stability. Loss of control of this process can lead to abnormal inflammatory responses and increased production of autoantibodies. RCP is associated with the endocytosis of macrophages. Rab protein is guanosine-5′-triphosphatase (GTPase) that can transport cellular debris and breakdown products to intracellular target compartments, thereby regulating the transport of substances in eukaryotic cells. Rab11 is a member of the Rab protein family and can bind to its interacting protein (FIP), which includes two types: type 1 and type 2. FIP type 1 can bind to Rab11 to form Rab11FIP1, i.e., RCP. RCP may be involved in FcγR-mediated phagocytosis and thus in the phagocytic process.

The existing mouse anti-RCP antibodies have no biological functions and cannot affect the phagocytic function of macrophages.

### SUMMARY OF THE INVENTION

The present invention provides an anti-RCP antibody that is biologically active and can reduce the phagocytic function of macrophages. The anti-RCP antibody of the present invention can be used to construct lupus-like mice.

In a first aspect, the present invention provides an anti-RCP antibody, including a heavy chain variable domain and a light chain variable domain;

the heavy chain variable domain including:
a heavy chain complementarity determining region HCDR1 consisting of the amino acid sequence of SEQ ID NO: 1,
a heavy chain complementarity determining region HCDR2 consisting of the amino acid sequence of SEQ ID NO: 2, and
a heavy chain complementarity determining region HCDR3 consisting of the amino acid sequence of SEQ ID NO: 3;
a light chain complementarity determining region LCDR1 consisting of the amino acid sequence of SEQ ID NO: 4,
a light chain complementarity determining region LCDR2 consisting of the amino acid sequence of SEQ ID NO: 5, and
a light chain complementarity determining region LCDR3 consisting of the amino acid sequence of SEQ ID NO: 6.

In the second aspect, the present invention provides a nucleic acid molecule encoding the anti-RCP antibody sequence as described above.

In the third aspect, the present invention provides a biological expression vector including the nucleic acid molecule as described above.

In the fourth aspect, the present invention provides a host cell including the biological expression vector as described above.

In a fifth aspect, the present invention provides a method for preparing an anti-RCP antibody, including the following steps:
prokaryotically expressing RCP isoform 2 protein, collecting the expressed fusion protein RCP-ECD-His, and purifying;
immunizing mice with the purified fusion protein RCP-ECD-His;
obtaining total RNA from B lymphocytes in the spleen of immunized mice, performing reverse transcription amplification to obtain cDNA sequences, and specifically amplifying mouse light chain antibody variable domain genes, light chain antibody constant domain genes, heavy chain antibody variable domain genes, and heavy chain antibody constant domain genes;
ligating the mouse light chain antibody variable domain genes, the light chain antibody constant domain genes, the heavy chain antibody variable domain genes, and the heavy chain antibody constant domain genes to vectors to obtain ligation products, and transforming the ligation products to construct a phage display library;
screening the phage display library to obtain an anti-RCP antibody sequence; and constructing an antibody protein expression vector using the anti-RCP antibody sequence, inducing expression, and purifying to obtain an anti-RCP antibody.

Compared to prior art, the present invention has the following advantageous effects. The anti-RCP antibody provided in the example of the present invention has biological functions and can reduce the phagocytic function of macrophages. The anti-RCP antibody provided in the example of the present invention can be used to construct lupus-like mice.

### BRIEF DESCRIPTION OF THE DRAWINGS

FIG. 1 shows the SDS-PAGE identification and analysis results of recombinant FIP protein;

FIG. 2 shows the SDS-PAGE analysis results of purified anti-RCP antibody; and

FIG. 3 shows the change in phagocytic function after the intervention of RAW264.7 by the anti-RCP antibody and Isotype group.

### DETAILED DESCRIPTION OF THE INVENTION

In this context, the expression of a range by “a number to another number” is a summary representation that avoids listing all values within the range one by one in the specification. Thus, the description of a particular numerical range encompasses any numerical value within that numerical range as well as any smaller numerical range defined by any numerical value within that numerical range, as if such numerical value and smaller numerical range were explicitly stated in the specification.

In this application, the use of “or” means “and/or” unless stated otherwise. In the case of multiple dependent claims, “or” is used merely in an alternative solution to reference more than one of the previous independent or dependent claims.

As used in accordance with the present disclosure, the following terms shall be understood to have the following meanings unless otherwise indicated.

It should be noted that the scientific and technical terms used in the present invention and their abbreviations have meanings commonly understood by those skilled in the art. Some of the terms and abbreviations used in the present invention are listed below:
antibody, heavy chain (HC), light chain (LC), heavy chain variable domain (VH), heavy chain constant domain (CH), light chain variable domain (VL), and light chain constant domain (CL). “Antibody” refers to a molecule that includes at least the complementarity determining regions CDR1, CDR2, and CDR3 of the heavy chain and at least CDR1, CDR2, and CDR3 of the light chain, which is capable of binding to an antigen. The term “antibody” includes, but is not limited to, fragments capable of binding to an antigen, such as Fv, single chain Fv (scFv), Fab, Fab′, and (Fab′) 2. The term “antibody” also includes, but is not limited to, chimeric antibodies, humanized antibodies, and antibodies from various species such as mice, humans, Cynomolgus macaques, and alpaca.

“Complementary determining region” or “CDR” is a region of an antibody variable domain that is highly variable in sequence and forms a structurally defined loop (“hypervariable loop”) and/or has an antigen-contacting residue (“antigen-contacting point”). CDRs are primarily responsible for the binding to antigenic epitopes. CDRs of the heavy and light chains are usually referred to as CDR1, CDR2, and CDR3, numbered sequentially from the N-terminus. CDRs located in the heavy chain variable domain of an antibody are referred to as HCDR1, HCDR2, and HCDR3. CDRs located in the light chain variable domain of an antibody are referred to as LCDR1, LCDR2, and LCDR3.

The present invention provides a method for preparing a mouse anti-RCP antibody, including the following steps:
1, synthesizing gene and constructing vector: 1) synthesizing a full-length gene sequence of RCP isoform 2 protein (645aa); 2) constructing the gene into a eukaryotic expression vector with a His tag;
2, expressing proteins: 1) constructing a transfection plasmid; 2) transfecting CHO cells and screening for high expression stable cell lines; 3) expanding the cells and expressing and purifying proteins of more than 2 mg for animal immunization and later screening; 4) performing SDS-PAGE identification analysis;
3, immunizing mouse: 1) immunizing 3 mice with RCP protein 4 times; 2) at the end of immunization, collecting serum samples to detect the titer;
4, preparing murine phage display monoclonal antibody: 1) selecting one mouse and removing its spleen to construct a mouse-derived phage display immune antibody library; 2) performing RCP protein selection, ELISA screening, and flow cytometry to screen Fabs that can affect the phagocytic function of macrophages, and sequencing; 3) expressing and purifying antibodies; and
5, expressing a full-length antibody: 1) selecting Fab antibodies to construct a mouse-derived full-length antibody expression vector; 2) preparing Expi-CHO cells for transfection; 3) completing transfection, expressing, and purifying.

The present invention will be described in detail below in connection with specific examples. It should be understood that these examples are only used to illustrate the present invention and are not intended to limit the scope of the present invention. In practical applications, modifications and variations made by those skilled in the art based on the present invention will still fall within the scope of the present invention.

### Example

This example provided an anti-RCP antibody, including a heavy chain variable domain and a light chain variable domain;

the heavy chain variable domain including:
a heavy chain complementarity determining region HCDR1 consisting of the amino acid sequence of SEQ ID NO: 1,
a heavy chain complementarity determining region HCDR2 consisting of the amino acid sequence of SEQ ID NO: 2, and
a heavy chain complementarity determining region HCDR3 consisting of the amino acid sequence of SEQ ID NO: 3;
a light chain complementarity determining region LCDR1 consisting of the amino acid sequence of SEQ ID NO: 4,
a light chain complementarity determining region LCDR2 consisting of the amino acid sequence of SEQ ID NO: 5, and
a light chain complementarity determining region LCDR3 consisting of the amino acid sequence of SEQ ID NO: 6.

| | Amino acid sequence |
|---|---|
| SEQ ID NO: 1 | GFTFSRYGMS |
| SEQ ID NO: 2 | TINSGGVYTY |
| SEQ ID NO: 3 | PPGNSAWFTY |
| SEQ ID NO: 4 | RASENIYSNLA |
| SEQ ID NO: 5 | GATNLAD |
| SEQ ID NO: 6 | QHFWGTPFT |

The light chain variable domain base sequence
(LC-NT) of the anti-RCP antibody provided in this
example (as shown in SEQ ID NO: 7) was as follows:
GACATTCAGATGACTCAGTCTCCAGTCTCCCTATCTGTATCTGTGGG
AGAAACTGTCACCATCACATOTCGAGCAAGTGAGAATATTTACAGTAATT
TAGCATGGTATCAGCAGAAACAGGGAAAATCTCCTCAGCTCCTGGTCTA
TGGTGCAACAAACTTAGCAGATGGTGTGCCATCAAGGTTCAGTGGCAGT
GGATCAGGCACACAGTATTCCCTCAAGATCAACAGCCTGCAGTCTGAAG
ATTTTGGGAGTTATTACTGTCAACATTTTTGGGGTACTCCATTCACGTTC
GGCTCGGGGACAAAGTTGGAAATAAAACGGGCTGATGCTGCACCAACTG
TATCCATCTTCCCACCATCCAGTGAGCAGTTAACATCTGGAGGTGCCTCA
GTCGTGTGCTTCTTGAACAACTTCTACCCCAAAGACATCAATGTCAAGT
GGAAGATTGATGGCAGTGAACGACAAAATGGCGTCCTGAACAGTTOGA
CTGATCAGGACAGCAAAGACAGCACCIACAGCATGAGCAGCACCCTCA
CGTTGACCAAGGACGAGTATGAACGACATAACAGCTATACCTGTGAGGC
CACTCACAAGACATCAACTTCACCCATTGTCAAGAGCTTCAACAGGAAT
GAGTGT.
The light chain variable domain amino acid
sequence (LC-AA) of the anti-RCP antibody provided
in this example (as shown in SEQ ID NO: 8) was as
follows:
DIQMTQSPVSLSVSVGETVTITCRASENIYSNLAWYQQKQGKSPQLLVYG
ATNLADGVPSRFSGSGSGTQYSLKINSLOSEDFGSYYCQHFWGTPFTFGS
GTKLEIKRADAAPTVSIFPPSSEQLTSGGASVVCFLNNFYPKDINVKWKI
DGSERQNGVLNSWTDQDSKDSTYSMSSTLTLTKDEYERHNSYTCEATHKT
STSPIVKSFNRNEC.
The heavy chain variable domain base sequence
(HC-NT) of the anti-RCP antibody provided in this
example (as shown in SEQ ID NO: 9) was as follows:
CAGGTGCAGCTGAAGGAGTCAGGGGGAGACTTAGTGAAGCCTGGA
GGGTCCCTGAAACTCTCCTGTGCAGCCTCTGGATTCACTTTCAGTAGATA
TGGCATGTCTTGGGTTCGCCAGACTCCAGACAAGAGGCTGGAGTGGGT
CGCAACCATTAATAGTGGTGGTGTTTACACCTACTATCCAGACAGTGTGA
AGGGGCGATTCACCATCTCCAGAGACAATGCCAAGAACGCCCTATACCT
GCAAATGAGCAGTCTGAAGTCTGAGGACACAGCCATGTATTACTGTGCA
AGGCCCCCTGGTAACTCGGCCTGGTTTACTTACTGGGGCCAAGGGACTC
TGGTCACTGTCTCTGCAGCCAAAACGACACCCCCATCTGTCTATCCACT
GGCCCCTGGATCTGCTGCCCAAACTAACTCCATGGTGACCCTGGGATGC
CTGGTCAAGGGCTATTTCCCTGAGCCAGTGACAGTGACCTGGAACTCTG
GATCCCTGTCCAGCGGTGTGCACACCTTCCCAGCTGTCCTGCAGTCTGA
CCTCTACACTCTGAGCAGCTCAGTGACTGTCCCCTCCAGCACCTGGCCC
AGCGAGACCGTCACCTGCAACGTTGCCCACCCGGCCAGCAGCACCAAG
GTGGACAAGAAAATTGTGCCCAGGGATTGT.
The heavy chain variable domain amino acid
sequence (HC-AA) of the anti-RCP antibody provided
in this example (as shown in SEQ ID NO: 10) was as
follows:
QVOLKESGGDLVKPGGSLKLSCAASGFTESRYGMSWVRQTPDKRLE
WVATINSGGVYTYYPDSVKGRFTISRDNAKNALYLOMSSLKSEDTAMYYC
ARPPGNSAWFTYWGQGTLVTVSAAKTTPPSVYPLAPGSAAQTNSMVTLGC
LVKGYFPEPVTVTWNSGSLSSGVHTFPAVLQSDLYTLSSSVTVPSSTWPSE
TVTCNVAHPASSTKVDKKIVPRDC.

In this example, the anti-RCP antibody, as described above, was constructed using the following methods.

### 1. Antigen Preparation, Identification, and Mouse Immunization

RCP isoform 2-ECD, an extracellular segment of RCP, was fused with 6*his and transiently expressed via an ExpiCHO system. The expression supernatant was centrifuged and subjected to affinity purification. The obtained protein sample was then determined for purity and concentration by SDS-PAGE. Only when the purity of the antigen reached 95% or above, and the antigen and antibody showed good binding, could it be used for subsequent animal immunization and antibody screening.

For the transient expression and purification of the antigen, the specific operation steps were as follows.

Plasmid preparation: a full-length gene was synthesized based on the sequence of the 6*his and RCP isoform 2-ECD fusion gene, and double restriction site sequences were introduced upstream and downstream of the gene to obtain gene fragments. The gene fragments were purified and recovered using 2% agarose gel, digested by double enzyme digestion with EcoRI (G/AATTC) and HindIII (A/AGCTT), and then respectively cloned into the pcDNA3.4 expression vector treated with the same enzyme digestion. The vector was transformed into E. coli. Monoclonal strains were screened. The plasmid was extracted for sequencing verification. The verified correct plasmid (the sequencing result was consistent with the original sequence) was used to transfect ExpiCHO-S cells in the next step.

On the day of transfection, ExpiCHO-S cells (purchased from Thermo Fisher, Cat No. A29127) had a density of 7×10⁶ to 1×10⁷ viable cells/mL and a cell viability of >98%, at which time the cells were adjusted to a final concentration of 6×10⁶ cells/mL with 25 mL of pre-heated fresh ExpiCHO expression medium at 37° C. The prepared plasmid (25 μg in total) was diluted with 1 mL of pre-cooled OptiPRO SFM at 4° C., and 80 μL of ExpiFectamine CHO was diluted with 920 μL of OptiPRO SFM. Then, the two were mixed well and gently blown to prepare an ExpiFectamine CHO/plasmid DNA mixture. The mixture was incubated at room temperature for 1-5 min, then transferred to the prepared cell suspension by slow addition while the cell suspension was gently shaken, and finally placed in a cell culture shaker for culture at 37° C. and 5% CO₂. ExpiCHO Enhancer and ExpiCHO Feed were added within 18 to 22 hours after transfection. The shake flask was placed on a shaker at 32° C. and 5% CO₂ to further culture. On day 5 after transfection, the same volume of ExpiCHO Feed was added slowly while the cell suspension was gently mixed well. Twelve to 15 days after transfection, the cell expression supernatant was centrifuged at high speed (15000 g, 10 min). The obtained His-tagged protein expression supernatant was subject to affinity purification through Ni Smart Beads 6FF (Changzhou Tiandiren & Biotechnology Co., Ltd., SA036050), and the protein of interest was then eluted with a gradient concentration of imidazole buffer. Finally, the obtained protein was replaced into PBS buffer through a concentration tube (Millipore, UFC901096).

The qualified RCP-ECD-His by purity identification was used for mouse immunization, and the specific operation steps were as follows. 6-8 weeks old female C57BL/6 mice (purchased from Shanghai Lingchang Biotechnology Co., Ltd.) were purchased. Immunization was performed by subcutaneous multi-site immunization, with the first immunization injection of 100 μg antigen and the second to fourth immunization injections of 50 μg antigen each time. The immunization was administered every two weeks for a total of four immunizations. The titer was determined by ELISA after four immunizations, with RCP-ECD-His used for an ELISA antigen-coated plate. The results showed that the titer reached 1:600000. At this time, 100 μg RCP-ECD-His was used for booster immunization. Two to 3 days later, the spleen was removed, and spleen B lymphocytes were isolated for subsequent construction of an immune antibody library.

### 2. Construction and Screening of Immune Antibody Library

B lymphocytes were isolated from the spleen of immunized mice obtained in the above step 1. RNA was extracted and reverse transcribed into cDNA using a reverse transcription kit (TaKaRa, 6210A). A series of primers were designed to amplify the variable domains and the first constant domains of the light and heavy chains, and the GIII protein of the M13 phage was fused to the C-terminus of heavy chain CH1. The obtained products were constructed into a phage display vector. The final constructed immune library was displayed as Fab on the coat protein of the M13 phage.

The specific operation steps were as follows.

Degenerate primers containing Ncol and AscI, Sfil and NotI restriction sites were designed based on the V genes and the first constant domain genes of all light and heavy chains in mice, respectively. PCR was performed to generate segments containing variable and constant domains. The antibody gene fragments of interest were inserted into a phage display vector by double enzyme digestion and ligation, in which the GIII gene was fused to the C-terminus of the heavy chain moiety of Fab. The ligation products were recovered by a recovery kit (Omega, D6492-02) and transformed into competent E. coli SS320 cells (Lucigen, MC1061F) by electroporator (BioRad, MicroPulser). The cells were thawed for 1 hour and then plated on an ampicillin-resistant 2-YT solid plate (prepared from 1.5% of tryptone, 1% of yeast extract, 0.5% of NaCl, and 1.5% of agar in mass/volume (g/mL)). For the calculation of storage capacity, 1 μL of bacterial solution was diluted and then plated on the plate for culture. The total number of colonies formed from all electrotransformation products, i.e., the storage capacity, was calculated by estimating the number of colonies grown. This immune library had a storage capacity of 1×10⁹ cfu.

Based on the storage capacity, 50 ODs (1 OD of 5×10⁸ cfu) of the mouse immune library bacteria constructed above were picked and added to a fresh 2-YT liquid medium. The bacteria were cultured at 37° C. and 220 rpm until a logarithmic growth phase. VSCM13 helper phage (purchased from Stratagene) was then added in an amount of 50 times the number of bacteria (i.e., the multiplicity of infection (MOI) of 50), mixed well, allowed to stand for 30 min, and further cultured on a shaker at 220 rpm for 1 hour. Subsequently, the culture was centrifuged at 10000 rpm for 5 min, and the supernatant was discarded. The culture solution was replaced with a 2-YT medium with dual resistance to carbenicillin/kanamycin, and the culture was further cultured at 30° C. and 220 rpm overnight. The next day, the bacterial solution was centrifuged at 13000 g for 10 min. The supernatant was collected and added with 20% PEG/NaCl (prepared from PEG 6000 with a volume concentration of 20% and 2.5 M NaCl) to achieve a final PEG/NaCl concentration of 4%, mixed well, placed on ice for 1 hour, and then centrifuged at 13000 g for 10 min. The phages corresponding to the mouse immune library obtained by precipitation were washed with PBS and then stored and used for subsequent phage screening.

The antibodies against RCP were screened by solid phase screening with immunotubes, and the specific method was as follows.

In the first round of screening, 4 mL of 50 μg/mL RCP-His was added to the immunotubes and coated at 4° C. overnight. The next day, the coating solution was discarded, and PBS containing 5% milk powder was added for blocking for 2 h. After washing twice with PBS, the prepared phages (the total amount of 1012 cfu) were added and incubated for 2 h. After washing 8 times with PBS and 2 times with PBS-T to remove non-specifically bound phages, 0.8 mL of trypsin digest solution containing 0.05% EDTA was added to the immunotubes to elute phages specifically binding to the target antigen. Next, the logarithmic phase SS320 bacteria (Lucigen, 60512-1) were infected with the obtained phages, allowed to stand at 37° C. for 30 min, and then cultured at 220 rpm for 1 h. After that, VSCM13 helper phage was added, allowed to stand for 30 min, and further cultured at 220 rpm for 1 h. The culture was centrifuged, replaced into C+/K+2-YT medium, and then further cultured at 30° C. and 220 rpm overnight.

The next day, the phages were prepared. The final obtained phages were further used in the second round of screening, repeating this process. The antigen concentrations for the second and third screenings were 5 μg/mL and 1 μg/mL, respectively. In addition, the PBS washing intensity gradually increased, and the PBS elution times were 12 and 16, respectively. Ten clones were randomly selected for sequence analysis in each round. After two or three rounds of screening, when the enrichment was obvious and the sequence showed high polymorphism, this round of bacteria was selected for monoclonal coating preparation. ELISA was performed on 0.1 mM IPTG-induced monoclonal Fab supernatant, and positive clones were selected for sequencing analysis. It was found that more than 60 positive clones with different sequences were obtained from this immune library screening. Fabs prepared from these clones were further incubated with RAW264.7 cells for 1 hour. Jurkat cells, a human T cell line, were induced to become apoptotic cells with staurosporine (purchased from Sangon Biotech (Shanghai) Co., Ltd.), stained with PHrodo (purchased from Life Technology) to obtain green fluorescence, and then phagocytosed with RAW264.7. The phagocytic function was then measured by flow cytometry. Among them, Fab with the most obvious decrease in phagocytic function was the target Fab.

### 3. Construction of Full-Length Mouse Anti-RCP Antibody

The Fab fragment constructed above was ligated to mouse IgG1 Fc. The ligation product was expressed and purified using the ExpiCHO transient expression system (Thermo Fisher, A29133). The specific method was described as step 1. Finally, the obtained antibody was replaced into PBS buffer through an ultrafiltration concentration tube (Millipore, UFC901096). The concentration was determined by an ultra-micro spectrophotometer (Hangzhou Allsheng Instruments Co., Ltd., Nano-300). The value obtained by dividing the measured A280 value by the theoretical extinction coefficient of the antibody was used as the antibody concentration value for subsequent studies. The antibody was dispensed and stored at −80° C.

### 4. The Anti-RCP Antibody can Affect the Phagocytic Function of Macrophages.

The anti-RCP antibody was incubated with RAW264.7 cells for 1 hour. Jurkat cells, a human T cell line, were induced to become apoptotic cells with staurosporine (purchased from Sangon Biotech (Shanghai) Co., Ltd.), stained with PHrodo (purchased from Life Technology) for 20 minutes to obtain green fluorescence, and then phagocytosed with RAW264.7 for 30 minutes. The phagocytic function of macrophages was then measured by flow cytometry. The results suggest that the anti-RCP antibody reduced the phagocytic function of macrophages compared to the Isotype group, i.e., the anti-RCP antibody provided in the present invention had biological functions.

The Isotype group was purchased from BBI, model Mouse IgG BBI D110503-0010.

It can be seen from FIG. 1 that under both non-reduced (NR) and reduced (R) conditions, 110 kDa and 75 kDa bands can be seen. Since the FIP protein contains five N-glycosylation sites, it is presumed that the two bands are both proteins of interest, and the difference in molecular weight results from glycosylation modification.

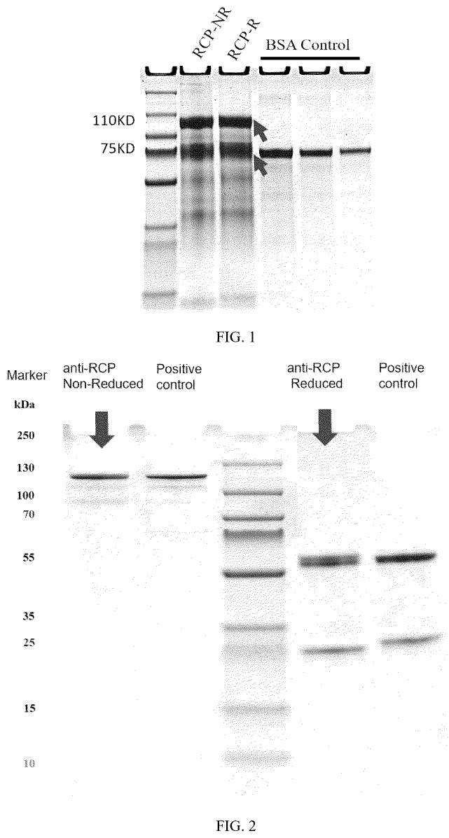

In FIG. 2, the left shows the expression of the anti-RCP antibody under a non-reduced (NR) condition, and the right shows the expression of the anti-RCP antibody under a reduced (R) condition.

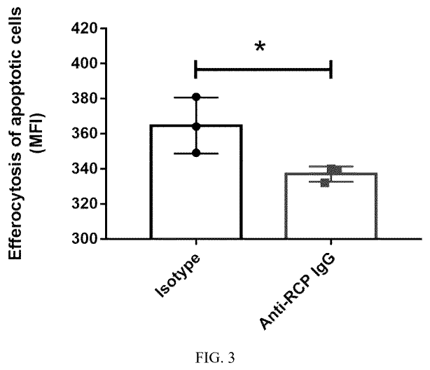

As can be seen from FIG. 3, the anti-RCP antibody can reduce the phagocytic function of mouse macrophages compared to the Isotype. The anti-RCP antibody prepared in the present invention can affect the phagocytic function of macrophages.

The examples disclosed above are only preferred examples of the present invention. Not all details are described in detail in the preferred examples, and the present invention is not limited to

1. An anti-RCP antibody, comprising a heavy chain variable domain and a light chain variable domain;
the heavy chain variable domain comprising:
a heavy chain complementarity determining region HCDR1 consisting of the amino acid sequence of SEQ ID NO: 1,
a heavy chain complementarity determining region HCDR2 consisting of the amino acid sequence of SEQ ID NO: 2, and
a heavy chain complementarity determining region HCDR3 consisting of the amino acid sequence of SEQ ID NO: 3;
the light chain variable domain comprising:
a light chain complementarity determining region LCDR1 consisting of the amino acid sequence of SEQ ID NO: 4,
a light chain complementarity determining region LCDR2 consisting of the amino acid sequence of SEQ ID NO: 5, and
a light chain complementarity determining region LCDR3 consisting of the amino acid sequence of SEQ ID NO: 6.

2. A nucleic acid molecule encoding the anti-RCP antibody according to claim 1.

3. A biological expression vector comprising the nucleic acid molecule according to claim 2.

4. A host cell comprising the biological expression vector according to claim 3.

5. A method for preparing an anti-RCP antibody, comprising the following steps:
prokaryotically expressing RCP isoform 2 protein, collecting the expressed fusion protein RCP-ECD-His, and purifying;
immunizing mice with the purified fusion protein RCP-ECD-His;
obtaining total RNA from B lymphocytes in the spleen of immunized mice, performing reverse transcription amplification to obtain cDNA sequences, and specifically amplifying mouse light chain antibody variable domain genes, light chain antibody constant domain genes, heavy chain antibody variable domain genes, and heavy chain antibody constant domain genes;
ligating the mouse light chain antibody variable domain genes, the light chain antibody constant domain genes, the heavy chain antibody variable domain genes, and the heavy chain antibody constant domain genes to vectors to obtain ligation products, and transforming the ligation products to construct a phage display library;
screening the phage display library to obtain an anti-RCP antibody sequence; and constructing an antibody protein expression vector using the anti-RCP antibody sequence, inducing expression, and purifying to obtain an anti-RCP antibody.


SEQUENCE LISTING (10 sequences)

SEQ ID NO: 1 - protein, 10 aa, unidentified
GFTFSRYGMS

SEQ ID NO: 2 - protein, 10 aa, synthetic construct
TINSGGVYTY

SEQ ID NO: 3 - protein, 10 aa, synthetic construct
PPGNSAWFTY

SEQ ID NO: 4 - protein, 11 aa, synthetic construct
RASENIYSNLA

SEQ ID NO: 5 - protein, 7 aa, synthetic construct
GATNLAD

SEQ ID NO: 6 - protein, 9 aa, synthetic construct
QHFWGTPFT

SEQ ID NO: 7 - DNA, 642 bp, synthetic construct
  source 1..642: mol_type = other DNA
gacattcagatgactcagtctccagtctccctatctgtatctgtgggagaaactgtcaccatcacatgtcgagcaagtgagaatatttacagtaatttagcatggtatcagcagaaacagggaaaatctcctcagctcctggtctatggtgcaacaaacttagcagatggtgtgccatcaaggttcagtggcagtggatcaggcacacagtattccctcaagatcaacagcctgcagtctgaagattttgggagttattactgtcaacatttttggggtactccattcacgttcggctcggggacaaagttggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgt

SEQ ID NO: 8 - protein, 214 aa, synthetic construct
DIQMTQSPVSLSVSVGETVTITCRASENIYSNLAWYQQKQGKSPQLLVYGATNLADGVPSRFSGSGSGTQYSLKINSLQSEDFGSYYCQHFWGTPFTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 9 - DNA, 663 bp, synthetic construct
  source 1..663: mol_type = other DNA
caggtgcagctgaaggagtcagggggagacttagtgaagcctggagggtccctgaaactctcctgtgcagcctctggattcactttcagtagatatggcatgtcttgggttcgccagactccagacaagaggctggagtgggtcgcaaccattaatagtggtggtgtttacacctactatccagacagtgtgaaggggcgattcaccatctccagagacaatgccaagaacgccctatacctgcaaatgagcagtctgaagtctgaggacacagccatgtattactgtgcaaggccccctggtaactcggcctggtttacttactggggccaagggactctggtcactgtctctgcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgt

SEQ ID NO: 10 - protein, 221 aa, synthetic construct
QVQLKESGGDLVKPGGSLKLSCAASGFTFSRYGMSWVRQTPDKRLEWVATINSGGVYTYYPDSVKGRFTISRDNAKNALYLQMSSLKSEDTAMYYCARPPGNSAWFTYWGQGTLVTVSAAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDC